(19) United States
(12) Patent Application Publication
(10) Pub. No.: US 2020/0238022 A1
(43) Pub. Date: Jul. 30, 2020

(54) APPARATUS FOR STIMULATING PATIENT TISSUE OR GUM INJECTION DURING MEDICAL PROCEDURE INJECTIONS

(71) Applicant: Ara Gulesserian, Glendale, CA (US)

(72) Inventor: Ara Gulesserian, Glendale, CA (US)

(21) Appl. No.: 16/672,263

(22) Filed: Nov. 1, 2019

Related U.S. Application Data
(60) Provisional application No. 62/755,294, filed on Nov. 2, 2018.

(51) Int. Cl. A61M 5/42; A61M 19/00

(52) U.S. Cl. CPC A61M 5/422; A61M 2205/586; A61M 2210/0631; A61M 19/00

(57) ABSTRACT

A gum numbing system includes a wristband configured to be attached to a user' wrist; an electronics housing; two or more finger coverings; two or more gum stimulation assemblies, the two or more gum stimulation assemblies detachably connected to the two or more finger coverings and the two or more gum stimulation assemblies to stimulate a patient's gum before a needle is inserted into the patient's gum; and two or more cables or wires, the two or more cables or wires connecting the electronics housing to the two or more gum stimulation assemblies. The electronics housing is integrated into the wristband or the top surface of the wristband.

### RELATED APPLICATIONS

This application claims priority to U.S. application Ser. No. 62/755,294, filed Nov. 2, 2018, entitled “Apparatus for Stimulating Patient Tissue or Gum Injection During Medical Procedure Injections,” the disclosure of which is hereby incorporated by reference.

### FIELD OF INVENTION

This disclosure relates to a method and apparatus for use in dental procedures, as well as other medical procedures, to reduce or minimize pain during injections of a liquid or an anesthetic into skin, tissue and/or gums of a patient.

### BACKGROUND OF THE INVENTION

In the prior art, the normal procedure for reducing the pain when injecting a liquid or other medical injections, such as, an anesthetic, serum, vitamins, vaccine, or liquids is either to (a) place a cold material against the skin, tissue or flesh of the patient at the injection site, (b) to apply a topical treatment to the skin, flesh or tissue at the injection site, which temporarily numbs the skin or flesh or (c) to manually massage the skin, flesh or tissue at a point of injection at the same time while performing the injection. These procedures are not particularly effective, because they require time to complete, are cumbersome and/or do not reduce the pain of injection to a satisfactory level.

FIG. 1 illustrates a retractor vibrator in the prior art (e.g., U.S. Pat. No. 9,675,766). The retractor vibrator 10 is a handheld apparatus comprising a main body or handle 12 in the shape of a tube having a battery cover and an end closure that is threaded to screw into an open end of a handle 12. In FIG. 1, a motor 24 drives a cam 26 housed in handle 12. The vibration induced by the cam and follower 100 fixed to rod 30 via a fitting 31 is transmitted via a light rode 30 to a tip 50. The vibrations are produced at free ends of the legs 42 of tip 50. This system was designed to attempt to reduce pain or discomfort during skin puncturing procedures including dentistry. This apparatus has the drawback of being a very rigid device that cannot contour to the tight spaces within the mouth. In addition, because this device is large, it is hard for a dentist to control this device along with controlling a needle for injection.

FIG. 2 illustrates a needle and an integrated vibration motor in the prior art. The control knob may be turned to activate a motor within the motor housing 210. The motor housing 210 includes a clip 215 which latches onto a barrel 220 of a dental needles or syringes. In embodiments, the motor in the motor housing 210 vibrates the barrel 220 (and thus the syringe or needle 225 attached thereto) when injections are administered to attempt to mitigate a patient's discomfort during dental injections. This apparatus has the drawback of vibrating a needle as it is being applied to the gum which may scare patients. In addition, the vibration of the needle may be dangerous as it enters the tissue or gum as it may move at the time of injection due to the vibration. In addition, the clip may become loosened which may lessen the vibration and thus the alleged mitigation of the discomfort for the patient.

Accordingly, a new system or device is needed to lessen or reduce the pain of injections into a gum or tissue of a patient, especially for dental patients that are receiving a shot of novocaine into areas of the gums which are incredibly painful.

### BRIEF DESCRIPTION OF THE DRAWINGS

FIG. 1 illustrates a retractor vibrator in the prior art;

FIG. 2 illustrates a needle and an integrated vibration motor in the prior art;

FIG. 3 illustrates a block diagram of a patient gum stimulation system according to embodiments;

FIG. 4A illustrates a glove stimulator including two or more vibration assembly holders or jackets according to embodiments;

FIG. 4B illustrates a side view of a vibration or stimulation assembly according to embodiments;

FIG. 4C illustrates a block diagram of a gum stimulation or vibration assembly shell according to embodiments;

FIG. 4D illustrates a block diagram of a gum stimulation or vibration assembly shell including a motor controller according to embodiments;

FIG. 4E illustrates a patient gum or tissue stimulation system utilizing a five-fingered glove according to some embodiments.

FIG. 5A illustrates a vibrating assembly utilizing a resonant circuit for generating a pulsed electro-magnetic field according to embodiments;

FIG. 5B illustrates a vibrating assembly utilizing a motor and a vibrating cap according to embodiments;

FIG. 5C illustrates a vibrating assembly utilizing a Transcutaneous Electrical Nerve Stimulation (TENS) unit or device to stimulate a gum area or a tissue area according to embodiments;

FIG. 5D illustrates a vibrating or stimulation assembly utilizing an ultrasound unit or device to stimulate a gum area or a tissue area according to embodiments;

FIG. 6A illustrates a block diagram of an embodiment of an electronics housing including a display according to some embodiments;

FIG. 6B illustrates a wearable computing device interfacing with two or more gum stimulation or vibrating devices according to some embodiments; and

FIG. 7 illustrates a block diagram of computing devices according to some embodiments.

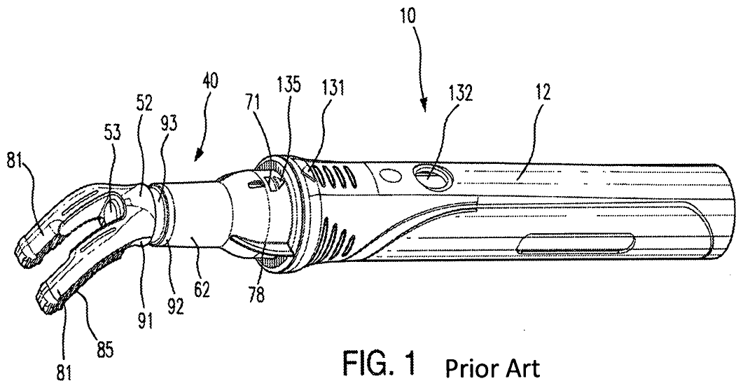

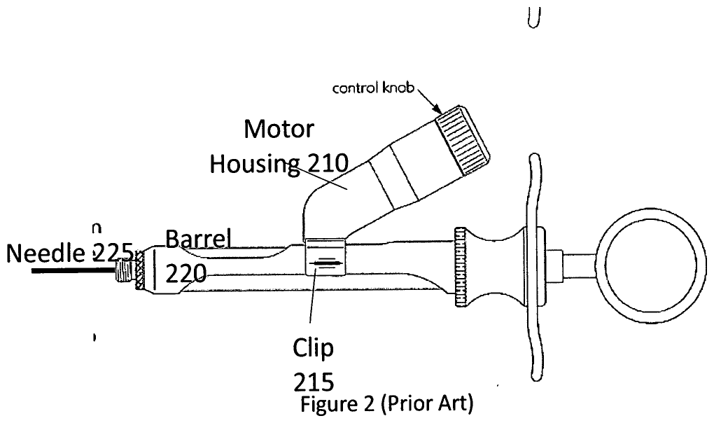

### DETAILED DESCRIPTION

The foregoing, and other features and advantages of the invention, will be apparent from the following, more particular description of the preferred embodiments of the invention, the accompanying drawings, and the claims.

In the following detailed description, numerous specific details are set forth to provide a thorough understanding of claimed subject matter. For purposes of explanation, specific numbers, systems and/or configurations are set forth, for example. However, it should be apparent to one skilled in the relevant art having benefit of this disclosure that claimed subject matter may be practiced without specific details. In other instances, well-known features may be omitted and/or simplified so as not to obscure claimed subject matter. While certain features have been illustrated and/or described herein, many modifications, substitutions, changes and/or equivalents may occur to those skilled in the art. It is, therefore, to be understood that appended claims are intended to cover any and all modifications and/or changes as fall within claimed subject matter.

References throughout this specification to one implementation, an implementation, one embodiment, embodiments, an embodiment and/or the like means that a particular feature, structure, and/or characteristic described in connection with a particular implementation and/or embodiment is included in at least one implementation and/or embodiment of claimed subject matter. Thus, appearances of such phrases, for example, in various places throughout this specification are not necessarily intended to refer to the same implementation or to any one particular implementation described. Furthermore, it is to be understood that particular features, structures, and/or characteristics described are capable of being combined in various ways in one or more implementations and, therefore, are within intended claim scope, for example. In general, of course, these and other issues vary with context. Therefore, particular context of description and/or usage provides helpful guidance regarding inferences to be drawn.

Likewise, in this context, the terms “coupled”, “connected,” and/or similar terms are used generically. It should be understood that these terms are not intended as synonyms. Rather, “connected” is used generically to indicate that two or more components, for example, are in direct physical, including electrical, contact; while, “coupled” is used generically to mean that two or more components are potentially in direct physical, including electrical, contact; however, “coupled” is also used generically to also mean that two or more components are not necessarily in direct contact, but nonetheless are able to co-operate and/or interact. The term “coupled” is also understood generically to mean indirectly connected, for example, in an appropriate context.

The terms, “and”, “or”, “and/or” and/or similar terms, as used herein, include a variety of meanings that also are expected to depend at least in part upon the particular context in which such terms are used. Typically, “or” if used to associate a list, such as A, B or C, is intended to mean A, B, and C, here used in the inclusive sense, as well as A, B or C, here used in the exclusive sense. In addition, the term “one or more” and/or similar terms is used to describe any feature, structure, and/or characteristic in the singular and/or is also used to describe a plurality and/or some other combination of features, structures and/or characteristics.

Likewise, the term “based on,” “based, at least in part on,” and/or similar terms (e.g., based at least in part on) are understood as not necessarily intending to convey an exclusive set of factors, but to allow for existence of additional factors not necessarily expressly described. Of course, for all of the foregoing, particular context of description and/or usage provides helpful guidance regarding inferences to be drawn. It should be noted that the following description merely provides one or more illustrative examples and claimed subject matter is not limited to these one or more illustrative examples; however, again, particular context of description and/or usage provides helpful guidance regarding inferences to be drawn.

Described herein is a unique and novel apparatus and method for reducing pain during injection or skin puncturing (or tissue or flesh puncturing) procedures, particularly involving dental injections. In many situations, a dentist or medical professional would like to be applying a stimulation technique to a gum or tissue area at a same time that an injection is being applied to the same general area of a gum or tissue. Thus, a dentist or doctor would want to be able to use one hand to control a needle for injecting a liquid or medication into a gum or tissue and utilize a second hand to control an apparatus or device for stimulating the tissue or gum to minimize the pain caused by the injection. In order to be utilized by a medical professional, an apparatus or device has to be simple to use and be able to be controlled utilizing one hand. In embodiments, a portion of the apparatus or device may need to be disposable because the portion may be placed in the patient's mouth. In addition, it is important for the gum or tissue to be stimulated on two sides. In embodiments, for example, utilizing a patient's gum as an example, the gum may be stimulated at positions that are on top and bottom of an injection site; positions that left or right of an injection site; and/or potentially stimulating next to a gum on an inside of a patient's mouth and stimulating outside of a patient's mouth at a position that is aligned to position inside a patient's mouth.

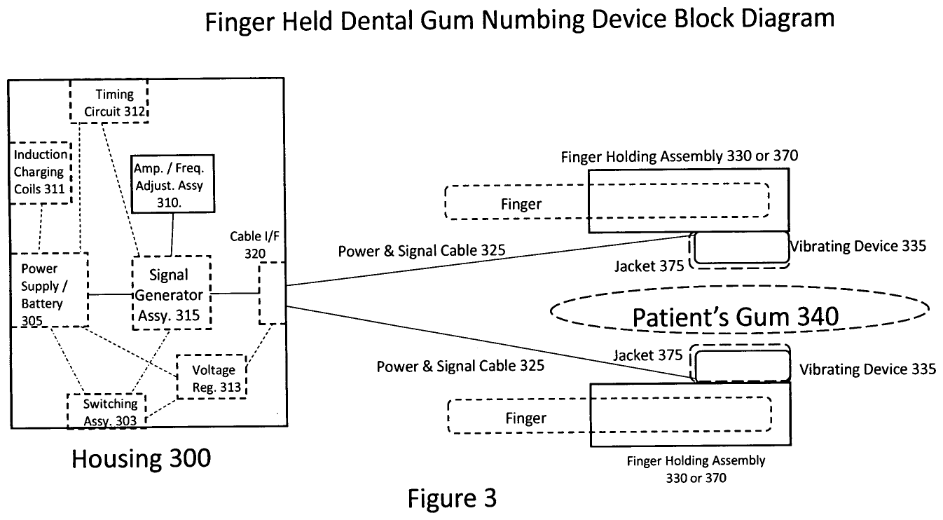

FIG. 3 illustrates a block diagram of a patient gum stimulation system according to embodiments. In the present application, claimed subject matter is directed to a patient gum stimulation system comprising a housing 300, one or more cables or wires 325, and/or two or more stimulation or vibration assemblies. In some embodiments, the housing 300 may comprise a switching assembly 303, a power supply 305, a signal generating assembly or apparatus 310, an amplitude and/or frequency adjusting circuit or assembly 315, and/or a cable or wire interface 320 (e.g., such as an interface port where cables or wires 325 may be inserted or plugged into). In some embodiments, a gum numbing system may further comprise one or more cables or wires 325, one or more finger vibration holding assemblies 330, and/or one or more vibrating devices 335. In the embodiment illustrated in FIG. 3, there are two or more cables or wires 325, two finger vibration holding assemblies 330, and two or more vibrating devices.

In some embodiments, a switching assembly 303 may be coupled to a power supply 305 to activate the power supply 305 and turn on or turn off a patient gum stimulation system. In some embodiments, the switching assembly 303 may be an on-off switch or a dial switch. In embodiments, the power supply 305 may supply a voltage and/or current to a signal generation assembly or device 310 in the housing 300 in response to connection via the switching assembly 303. Thus, in some embodiments, the power supply 305 may be a rechargeable power source, such as a rechargeable battery. In some embodiments, the switching assembly 303 may activate a signal generator assembly 315 in order for a signal to be generated and/or transmitted to the vibrating devices 335. In some embodiments, the signal generation device 310 receive the input voltage and/or current and may generate a signal with a specific amplitude and/or a specific frequency of a voltage and/or current. In some embodiments, a signal generation device 310 may generate a DC current or voltage. In some embodiments, a user and/or operator may determine and/or select the amplitude and/or frequency via a frequency and/or amplitude adjusting circuit 315 (e.g., this may be optional). In embodiments, a knob, an adjustable switch and/or a button may allow adjustment of the amplitude and/or frequency via utilization with and/or connection to a signal adjusting circuit 310. In some embodiments, a signal adjusting circuit 310 may include a potentiometer. In some embodiments, the signal adjusting circuit may be an automatic gain control circuit, e.g., a closed-loop feedback regulating circuit. In some embodiments, the generated signal may be a DC signal or may be an AC signal. In embodiments, the generated signal may be a square wave, a triangular wave, a pulsing waveform or a sinusoidal waveform. The signal generator assembly or circuitry 315 may be any signal generators such as those made by Texas Instruments, Analog Devices, or other manufacturers. In embodiments, the signal generation assembly 315 may transfer and/or communicate the generated signal to the cable and/or wire interface 320 in the housing 300. In embodiments, the cable and/or wire interface 320 may comprise two or more jacks to which cable connectors or wire connectors (and thus a cable or one or more wires 325) may be coupled and/or connected.

In embodiments, the cable and/or wire interface 320 may allow for detachable connections so that the two or more wires or cables 325 may be detached from the housing 300 when not in use by a medical professional. It will be important for the patient gum stimulation system to be portable and be able to be stored in a separate housing or case so that a dentist or medical practitioner may carry portions and/or all of a patient gum stimulation system in a medical practitioner's uniform shirt pocket and/or or a pants pocket. In other words, the patient gum stimulation system may be portable and/or capable of being carried in a small case. In addition, it would be advantageous for the housing 300 to be able to be mounted to a user's arm or a user's waist. For example, the housing 300 may be attached to and/or part of a wristband, an arm band, a belt, and/or a pocket. In some embodiments, the housing 300 may be small in size. For example, sample housing sizes may be the size of a wrist watch or an Apple Watch. In some embodiments, a housing may be 1 inch length and 1 inch in width. In embodiments, a housing may have a length in the range of 0.5 inches to 10 inches and a width in the range of 0.5 inches to 10 inches. In embodiments, a housing 300 may have a thickness similar to that of a credit card. In embodiments, a housing 300 may have a thickness of ¼ inch, ½ inch or greater than ½ inch. In some embodiments, the housing 300 may have a thickness ranging from 1/16 of an inch to 3 or 4 inches. If the housing is oval or circular, the housing may have a diameter of approximately 1 inch or may have a diameter ranging from 0.5 inches to 8 inches. These dimensions may be representative of any of the other drawings included within this patent application.

In some embodiments, the housing 300 may further comprise a timing circuit 312. In some embodiments, this turns on and/or activates the signal generator assembly or circuit 315 and/or the power supply battery 305 for a specific period of time and thus the voltage, current and/or signal is transmitted or provided only for that specific period of time. In some embodiments, the timing circuit may be a real-time clock and/or an oscillator circuit. In some embodiments, the housing 300 may further induction charging coils 311. In some embodiments, the induction charging coils 311 may be able to wireless receive power from a wireless charging pad and/or induction charging apparatus. In other words, the housing 300 may be placed on a wireless charging pad or appropriate surface of the induction charging apparatus (including the wireless charging coils) and the induction charging coils 311 may create a voltage that is supplied to the power source/battery 305. In some embodiments, the housing 300 may comprise a voltage regulator 313. In some embodiments, the voltage regulator 313 may work in concert or together with the power supply or battery 305 and/or switching assembly 303 in order to provide a specific regulated voltage and/or current to the two or more vibrating devices 335. In this embodiment, for example, when a switching assembly 303 activates or provides a path to the voltage regulator 313 (e.g., or turns on the voltage regulator 313), the voltage regulator 313 may transmit the desired voltage to the two or more vibrating devices 335 through the cable interface or port 325 and/or the associated two or more cables or wires 325.

In some embodiments, the two or more cables or wires 325 may connect at another end (e.g., a distal end from the housing) to two or more stimulation or vibration assemblies. In some embodiments, the two or more patient vibration or stimulation assemblies may each include a finger holding assembly 330, a jacket 375 and/or a vibrating device 335. In some embodiments, there needs to be two or more vibration stimulation or vibrating assemblies so that the stimulation or vibration assemblies may be placed on opposite sides of a needle injection site or a medication injection site. In some embodiments, in order for one hand to operate the two or more stimulation or vibration assemblies, the two or more vibration devices 335 may be placed inside of and/or attached to two or more finger gripping/insertion housings and/or fabrics holding assemblies 330. In some embodiments, this allows two fingers to be attached or connected to associated or corresponding vibration devices 335. In some embodiments, the vibration devices 335 may be placed inside a jacket 375 to protect the patient's gum or tissue or saliva (or other liquid or bacteria) from touching the vibration devices 335. In some embodiments, this allows a medical professional to only have to utilize two fingers (and not the whole hand) for stimulating a patient's gum or tissues during a medical injunction and to have other fingers available for performing other actions if necessary. In some embodiments, a finger gripping or holding housing 330 may have two portions or sections. In embodiments, a finger holding housing may have a finger insertion section 370 and a vibration assembly holder section or jacket 375. In some embodiments, this allows a medical professional or doctor to control utilization of the vibration and stimulation assemblies independently (e.g., each vibration device is coupled to and/or controlled by a medical professional's finger and thus can be operated independently of other vibration and stimulation assemblies). In some embodiments, this allows the entire patient gum or stimulation system to easily be placed in a carrying case. In this embodiment, the finger holding housings or assemblies 330 may be separate from each other (e.g., not part of another material and not connected to another finger gripping housing). While the benefits of the gum or tissue stimulation device may still be achieved utilizing such a configuration, such a configuration may be difficult to keep track of because the finger insertion sections are separate from each other along with being separate from the cables and/or wires 325. A medical professional may have too many separate pieces that are too difficult to keep track of. In embodiments, this problem may be solved by utilizing a glove to connect to connect different finger insertion or holding housings 330 and have one clothing item that needs to be worn that includes everything necessary to provide a gum stimulation device.

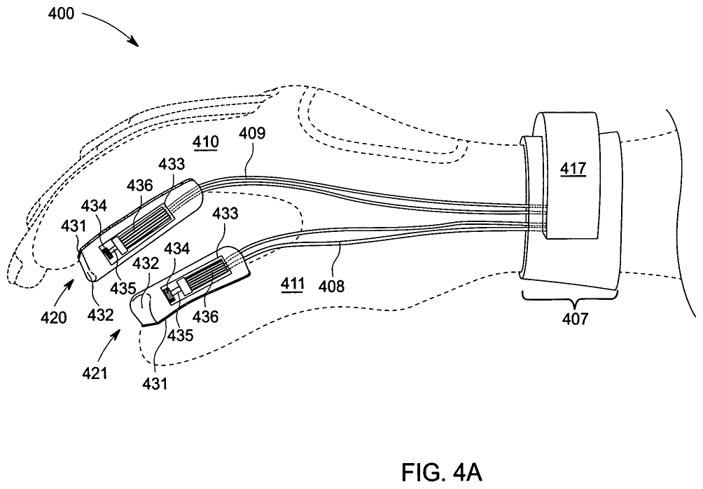

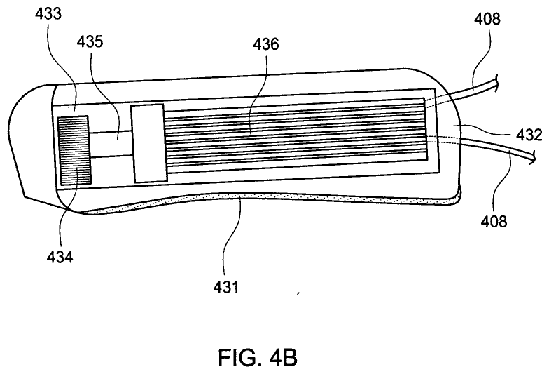

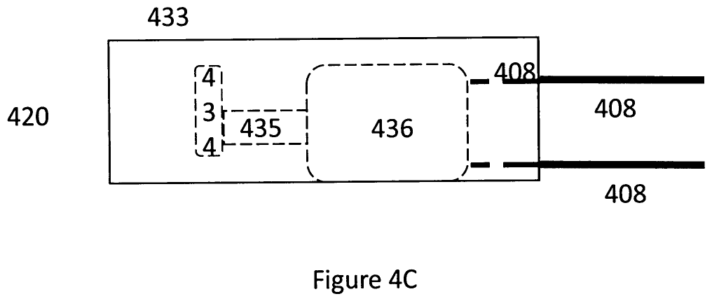

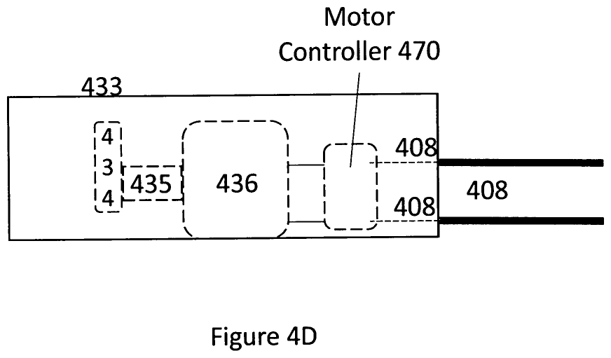

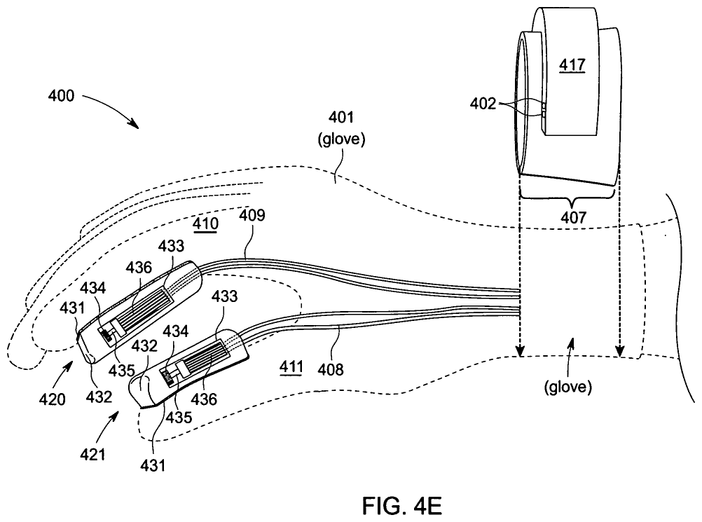

FIG. 4A illustrates a glove stimulator including two or more vibration assembly holders or jackets according to some embodiments. FIG. 4B illustrates a side view of a vibration or stimulation assembly according to some embodiments. In some embodiments, a glove stimulator may be a two fingered glove, which may be made of a variety of fabrics and specifically for use as part of a gum or tissue stimulation system including a vibration or stimulation assembly. In embodiments, the two fingered glove may be a medical glove or thin material glove. In embodiments, a two fingered glove may be utilized since in some cases, only a thumb and index finger of a medical professional's hand may be utilized in utilizing the vibration assembly holders or jackets according to embodiments. In some embodiments, medical gloves may be made of different polymer materials including latex, nitrile rubber, polyvinyl chloride and neoprene. In some embodiments, the medical gloves may be full five fingered gloves. In some embodiments, existing medical gloves may be utilized with gum or tissue stimulation system.

In some embodiments, a glove tissue or gum stimulation system 400 may comprise a wristband 407, an electronics housing 417, two or more cables or wires (or sets of cables or wires) 408 409, two or more finger coverings 410 411, and/or two or more stimulation or vibration assemblies 420 421. In some embodiments, a glove tissue or gum stimulation system 400 may further comprise an adhesive layer 431. In some embodiments, an electronics housing 417 may be integrated with the wristband 407. In some embodiments, an electronics housing 417 may be attached to the wristband 407 and/or may reside on a top surface of the wristband 407 (or alternatively to another surface of the wristband 407). In some embodiments, an electronics housing 417 may comprise a rechargeable power source, one or more signal generators and/or one or more cable or wire interface ports (as discussed above and illustrated in FIG. 3). In some embodiments, the electronics housing 417 may comprise similar components to the housing 300 described above in FIG. 3. In some embodiments, the batteries or power sources of the electronics housing 417 may be rechargeable. In some embodiments, the electronics housing 417 may be detachable to allow the rechargeable power sources to be recharged separately via an induction charging plate or via a charging cord (e.g., the electronics housing 417 may have a USB interface for charging purposes and/or an induction charging coil for charging purposes). In some embodiments, the batteries or power sources of the electronics housing 417 may be detachable from the housing so that they may be replaced or recharged external to the electronics housing 417. In some embodiments, for example, the electronics housing 417 (and power source) may include one or more batteries, a switching assembly, an adjustment assembly, and a power interface port.

In some embodiments, the power source (e.g., battery) may provide power signals and/or control signals to the power interface port (or interface) so that power may be transferred or transmitted out of the power and signal interface port (or interface) to the stimulation or vibration assemblies 420 421 via the cables or wires 408 409. In some embodiments, different signals may be communicated to the stimulation or vibration assemblies 420 421 via the cables or wires 408. In some embodiments, these may be square waves, triangular waves, sinusoidal waves, or other waveforms. In some embodiments, a switching assembly (e.g., a button or press switch) may be utilized to control a transfer of power and/or signals to the stimulation or vibration assemblies 420 421. In other words, the switching assembly may control whether or not the signals are transmitted to the stimulation or vibration assemblies 420 421. In some embodiments, the power signals and/or control signals may be a DC voltage signal having one or more amplitudes and/or one or more frequencies. In some embodiments, the electronics housing 407 may include a button and/or a press switch to open or close a circuit of the switching assembly and thus turn on and/or off the providing of power signals or control signals to the stimulation or vibration assemblies 420 421. In embodiments, the electronics housing 407 may include a knob or dial (e.g., an adjustment assembly) to adjust an amplitude of power or a frequency of a power signal or control signal provided to the stimulation or vibration assemblies 420 421 through the cables or wires 408 409. By varying an amplitude and/or a frequency of a power and/or control signal to the stimulation or vibration assemblies 420 421, the electronics housing 407 may cause a motor in the stimulation or vibration assemblies 420 421 to rotate or spin at a faster rate (or vice versa, rotate or spin at a slower rate).

In some embodiments, a medical professional's finger may be placed or inserted into associated or corresponding finger coverings 410 or 411. In some embodiments, the associated or corresponding finger coverings 410 or 411 may be part of the glove (e.g., a full hand glove) or two-fingered gloves. In some embodiments, an injection or application site for the needle may be located or positioned between the two fingers (and thus the finger coverings 410 and 411). In some embodiments, one finger or a medical professional may be placed on an inner surface of a patient's mouth and the other finger may be placed on a surface of a patient's cheek. Either of the fingers and/or vibration assemblies 420 421 may be pressed against the check or inner lining of a patient's mouth to stretch the inner lining of the patient's mouth with the respect to the outer cheek of the patient's mouth in order to minimize patient discomfort (or one or two of the fingers may apply a pressure to stretch the gum or tissue). In some embodiments, the two or more stimulation or vibration assemblies 420 421 may be placed or inserted into the two or more stimulation jacket housings (which are similar to the jackets 375 illustrated in FIG. 3). In some embodiments, one or more stimulation or vibration assemblies 420 421 may be attached or adhered to an outer surface of finger coverings 410 or 411. In some embodiments, one or more stimulation assemblies 420 421 may comprise an adhesive or adhesive layer 431 to attach or adhere to an outer surface of a glove (e.g., the finger coverings 410 or 411 of the glove). In some embodiments, one of the fingers in the glove may be a medical provider's thumb and a second one of the fingers in the glove may be an index finger. Thus, in some embodiments, where the one or more stimulation or vibration assemblies 420 421 are attached or adhered to an outer surface of the glove via an adhesive layer 431, the one or more stimulation or vibration assemblies 420 421 may be attached to an inner surface of a glove finger covering a medical provider's thumb and an inner surface of a glove finger covering a medical provider's index finger.

In some embodiments, the one or more stimulation or vibration assemblies 420 421 may comprise an adhesive layer 431, a covering 432, a shell 433, a cap 434, a shaft 435 and/or a motor 436. In some embodiments, the adhesive layer 431 may adhere a covering 432 of a stimulation or vibration assemblies 420 421 to a glove surface (e.g., a surface or an inside surface of a glove finger 410 411). In some embodiments, the adhesive layer 431 may be a glue, a sticky substance, or a tacky substance that will not tear the covering or tear the glove, but may allow the stimulation or vibration assemblies 420 421 to be adhered to the finger coverings 410 411 or a glove surface. In some embodiments, the gloves themselves (or glove finger coverings 410 411) may have a sticky substance or adhesive substance or layer which may allow the one or more stimulation or vibration assemblies 420 421 to attach or adhere to the gloves. In some embodiments, a covering 432 may encompass or encircle a shell 433 of the stimulation or vibration assemblies 420 421. In embodiments, a covering 432 may be a soft plastic, a paper or a cloth surface. In embodiments, a covering 432 may be made of a similar material to a glove material. In embodiments, a shell 433 may be made of plastic, a light metal or a composite material. In embodiments, a shell may be range from 0.5 to 4 inches in length and may have sides of 0.1 inches to 2 inches in width (or a diameter of 0.1 to 2 inches). In some embodiments, a shell 433 may be shaped like a long oval or a bullet. In embodiments, a covering may also have similar dimensions. In embodiments, a shell 433 and/or a covering 431 may have other shapes or sizes that may still comfortably inserted into a patient's mouth and/or be placed on a patient's cheek in order to stimulate the gum before a needle is inserted.

In embodiments, a motor 436 in a stimulation or vibration assembly 420 421 may receive power (e.g., DC power and/or control signals) via one or the wires or cables 408 or 409. In embodiments, when a motor 436 receives power, a motor 436 is activated or turned on and rotates a shaft 435, which in turn rotates a cap 434. In embodiments, the rotation of the cap 434 against the shell 433 causes the stimulation or vibration assemblies 420 421 to vibrate. In embodiments, the motor 436 and/or the cap 434 may also vibrate, which also may generate additional vibration movement of the stimulation or vibration assemblies 420 421. In some embodiments, the shaft 435 and/or cap 434 may rotate at 20 revolutions per minute, 40 revolutions per minute, 60 revolutions per minute, 120 revolution per minute or 240 revolutions per minute (or another value of rpm up to 800 rpm). In some embodiments, a rotation speed of a motor 436 may be determined based at least in part on an amplitude of input signal communicated via the one or more wires or cables 408 or 409. In some embodiments, input signals may be pulse width modulated where a frequency of the pulses may determine a rotation speed of the motor 436. In embodiments, input signals may be pulse width modulated where a width of pulses may determine a rotation speed of the motor 436. In embodiments, other uses of signal modulation may be utilized to control rotational speed of a motor 436. In embodiments, two wires may be utilized in the wires or cables 408 409 that deliver the power and/or control to the motor 436 and in other embodiments, three-wire, four-wire or six-wire cables may be utilized to deliver power and/or control signals to the motor 436. In embodiments, amplitudes of input signals may determine an amplitude of a rotation speed of a motor 436. In embodiments, input signals may be DC voltage or current signals, control signals, and/or AC voltage or current signals.

In some embodiments, finger covering 410 may be a thumb finger covering and finger covering 411 may be an index finger covering. Similarly, in some embodiments, including a five-fingered medical glove, the stimulation or vibration assemblies 420 421 may be attached or adhered to a surface of a thumb part of the medical glove and/or an surface of an index finger part of the medical glove. in illustrative embodiments, stimulation or vibration assembly 420 may be placed on an inside surface of a medical provider's thumb. In some embodiments, the stimulation or vibration assembly 421 may be placed on an inside surface of a medical provider's index finger.

In some embodiments, a gum stimulation or vibration assembly 420 or 421 may be disposable. In other words, a medical professional such as a dentist may utilize the gum stimulation or vibration assembly 420 or 421 once and because these devices will be utilized in a patient's mouth, the devices may be disposable and placed into the trash. In embodiments, the wires or cables 408 or 409 may detach from the associated gum or vibration assembly 420 to allow the associated gum or vibration assembly 420 421 to be easily thrown away.

In some embodiments, the motors 436, shaft 435 and cap 434 that are part of the gum and vibration assembly may be too expensive to dispose each time an injection into a gum or tissue is completed. In these cases, a covering 432 and/or adhesive layer 431 may be disposable and may be discarded each time an injection is completed. This may be easier and in some embodiments, the covering 432 may have a zipper, a button, or may be tearable to allow the shell 433 (and motor 436, shaft 435 and cap 434) to be easily removed so that they can be disposed easily. In this embodiment, the covering 432 and/or adhesive layer 431 may then be thrown away or disposed. In some embodiments, the glove may also be thrown away or disposed. This may be an option, because in this embodiment, the shell 433 (and motor 436, shaft 435 and cap 434) may never touch a patient's skin or a patient's gum or mouth due to the covering 432. Nonetheless, in some embodiments, the shell 433 may be waterproof so as to allow the shell 433 to be sterilized by the medical professional. In other words, no water may be able to enter the shell and interfere with operation of the motor 436, shaft 435 and cap 434). In some embodiments, a shell 433 may be sterilized and/or washed.

FIG. 4C illustrates a block diagram of a gum stimulation or vibration assembly shell according to some embodiments. FIG. 4D illustrates a block diagram of a gum stimulation or vibration assembly shell including a motor controller according to some embodiments. In some embodiments, a gum stimulation or vibration assembly shell 433 may comprise a motor assembly 436, a motor shaft 435 and a rotating cap 434. In embodiments, a power signal and/or signal control may be transferred or transmitted via two or more wires or cables 408 to the gum stimulation or vibration assembly 433. As discussed previously, the gum stimulation or vibration assembly shell 433 may be encapsulated or enclosed within a covering (not shown in FIG. 4C). In embodiments, when activated or turned on, the motor assembly 436 may operate and may rotate a motor shaft 435, which in turn may rotate a cap 434. In embodiments, the rotation of the motor shaft 435 and the cap 434 may cause the gum stimulation or vibration assembly shell to vibrate. In embodiments, the motor 436, motor shaft 435 and/or cap 434 may themselves also vibrate to add to the vibration of the shell. In some embodiments, the vibration of the gum assembly causes the patient's gum to be stimulated.

In some embodiments, the motor speed (and thus the rotation speed of the motor shaft 435 and/or the cap) may be varied and/or may be adjustable. In embodiments, the signals may be varied in amplitude, frequency, and/or period in order to change the motor's 436 speed and thus vary a number or rotations of the motor shaft 435 and/or a cap 434. In some embodiments, this is in turn causes a change in vibration amplitude of a gum stimulation or vibration assembly shell 433 and a change in stimulation of a patient's gum. In embodiments, a vibration or stimulation assembly shell 433 may further comprise a motor controller 470. FIG. 4E illustrates a patient gum or tissue stimulation system where a five-fingered glove 401 is utilized (e.g., utilizing a normal medical provider's glove). FIG. 4E also illustrates how the armband may be inserted onto an arm or wrist of a medical processional. FIG. 4E further illustrates an electronic housing 417 having one or more output signal ports for the one or more cables 408 or 409 to connect to.

In some embodiments, the rechargeable power source, a signal generating device, a switching assembly and/or other components that are currently in the electronics housing may small enough to be placed in the one or more stimulation or vibration assemblies. In these illustrative embodiments, there is no use of external cables because the one or more stimulation or vibration assemblies may include all the components and assemblies necessary to provide power and to vibrate the assembly. In this embodiment, a covering may be utilized to protect the stimulation or vibrating assemblies from bacteria, saliva and/or pathogens of the patient and/or that are existing in the air. As described above, an adhesive layer may connect these stimulation or vibration assemblies (that have all the necessary components integrated) to a medical professional's glove.

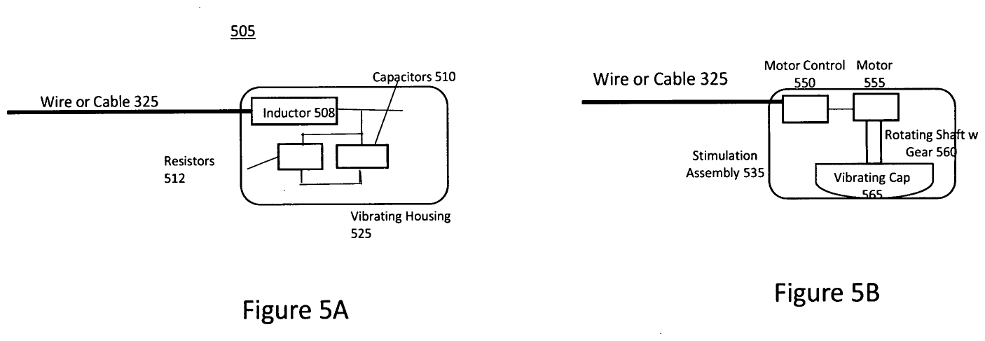

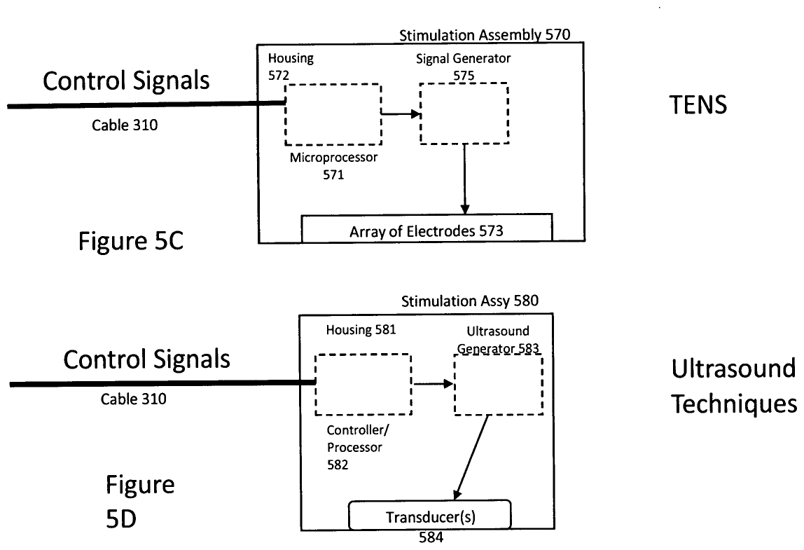

In some embodiments, there are alternative embodiments for providing stimulation to the gum or tissue of the patient other than those discussed above with respect to FIGS. 4A to 4E. In some embodiments, a stimulation or vibration device may utilize: 1) a resonant circuit to create vibration and/or stimulation; 2) a motor, shaft and vibrating cap to create vibration and/or stimulation at the injection site, 3) a transcutaneous electrical nerve stimulation (TENS) unit to create vibration and/or stimulation at the injection site; and/or 4) an ultrasound unit to provide vibration or stimulation to the gum or tissue of the patient at the injection site. Each of the devices describe in FIGS. 5A-5D may be encapsulated in shell as described above with respect to FIGS. 4A-4E. As discussed above, these different embodiments (e.g., described in FIGS. 5A-5D), may also include power sources and/or switching assemblies in order to be completely powered without having a separate electronics housing and/or wristband. In other words, a resonant circuit-based vibrating assembly, a motor controller and rotating cap vibrating assembly, a TENS unit vibrating assembly and/or an ultrasound vibrating or stimulation assembly may be encapsulated in a shell; may have a covering surrounding a shell; may be small enough to be adhered to fingers of a two-fingered glove and/or medical glove, may have a detachable port for attaching to wires and receiving power signals and control signals; and may or may not be disposable. In addition, these units may also be self-contained in the future and may not need wires or cables for operation (e.g., all components and/or assemblies may be in the housing or shell).

FIG. 5A illustrates a vibrating assembly utilizing a resonant circuit for generating a pulsed electro-magnetic field according to embodiments. In some embodiments, a vibration assembly or stimulating assembly 505 may comprise a housing 525. In some embodiments, a housing 525 may be made of a plastic material, a composite material, a glass or quartz material. In some embodiments, a housing 525 may be circular, oval, square or rectangular in shape. In embodiments, a housing 525 may include one or more inductors or inducting coil 508, one or more resistors 512 and/or one or more capacitors 510. In some embodiments, a signal generation device or signal generator 310 (FIG. 3) may generate a signal and/or waveform and thus may supply a current and/or voltage via a wire and cable 325 to the housing 525. In some embodiments, the signal and/or waveform may cause the stimulation device or assembly 505 to generate a magnetic field. In some embodiments, the stimulation device or assembly 505 may apply a low strength, low frequency magnetic field wave to a patient's tissues and/or gums. In some embodiments, the supplied current and/or voltage from the signal generator may excite the one or more inducting coils 508, which in term may generate a magnetic field that gently vibrates or soothes the gum or tissue. In some embodiments, the one or more resistors 512 or capacitors 510 may filter and/or received signal. In embodiments, the one or more inductor coils 508 may be a 16 turn single layer coil (including copper wire around a hollow core) in series with one or more resistors 512 (which are in parallel with one or more capacitors 510). In some embodiments, the application of the current may generate an oscillating magnetic field surrounding the conductive coil. In some embodiments, the housing 525 may surround the conductive and inductive coil (to keep the coil from touching the gum or tissue) but still allow penetration of the magnetic field emitted from the conductive coil into the tissue. In some embodiments, an inductive coil 508 may have between 1-300 turns, may have a diameter of 0.3 to 30 millimeters, and/or may generated a magnetic field strength of 0.005-15 m Tesla near the inductive coil 508. In some embodiments, a current generated may have a pulse frequency of 0.1 to 1,000 Hertz. In some embodiments, a current generated may have a frequency range of 5 Hz to 100 kilohertz. In some embodiments, the resistors 512 and capacitors 510 may form a filter to filter the incoming current supplied to the inducting coil 508. In embodiments, a stimulation device or assembly 505 may be further include an electromagnetic vibration motor. In embodiments, the incoming current may cause the turning on and/or vibration of the electromagnetic vibration motor. In embodiments, the electromagnetic vibration motor may be placed next to the tissue or gum of the patient and may deliver both a vibration and/or an accompanying magnetic emission into the patient's tissue or gum.

FIG. 5B illustrates a vibrating assembly utilizing a motor and a vibrating cap according to embodiments. In some embodiments, a vibrating or stimulating assembly 535 may comprise a motor controller 550, a motor 555, a rotating shaft with a gear assembly 560 and/or a vibrating cap or plastic assembly 565. In some embodiments, the signal generation device or signal generator 310 may communicate one or more signals through the one or more wires or cables 325 to the motor controller 550 to activate the motor controller 550. In embodiments, the one or more signals may be activation signals. In embodiments, the one or more signals may also include control signals which include information such as amplitude, frequency and/or intensity parameters and/or values of a signal that is to be communicated to the motor 555. In some embodiments, the motor controller 550 may communicate signals and/or instructions to the motor 545 to activate or turn on. In some embodiments, the motor 545 may rotate a shaft 560 having a gear at a distal or another end. In some embodiments, a vibrating cap or plastic assembly 565 may be connected to the distal or another end of the shaft 560 including the gear. In embodiments, the vibrating cap or plastic assembly 565 may have a receptacle that mates with or is attached to the gear (which is part of the shaft 560). In some embodiments, rotation of the shaft causes the gearing assembly to rotate and cap or plastic assembly begins to move and/or vibrate due to the rotation of the shaft or gearing assembly 560. If the gearing assembly 560 symmetrically mates with a receptacle in the cap or plastic assembly 565, the vibration of the cap or plastic assembly may be controlled. In some embodiments, the gearing assembly may not be a perfect fit in the cap or plastic assembly, and thus the vibration may not be uniform. In some embodiments, the cap or plastic assembly 565 may press against a skin or cover of the vibrating or stimulating assembly. In some embodiments, the skin or cover may be a fabric, may be a soft plastic material and/or may be a composite material. In embodiments, the vibrating or stimulating assembly 535 may be small enough to be positioned or installed in a jacket that is attached to a glove finger.

FIG. 5C illustrates a vibrating assembly utilizing a Transcutaneous Electrical Nerve Stimulation (TENS) unit or device to stimulate a gum area or a tissue area according to embodiments. In embodiments, a TENS unit or device may be utilized to stimulate and/or merge nerve endings in a patient's gums or tissues and thus reduce and/or eliminate pain. In embodiments, a TENS unit may generate a waveform may have an output intensity and/or amplitude of 0-50 mA, although in some embodiments, the TENs unit may generate a waveform having an output intensity or waveform of 0 to 80 mA. In embodiments, a TENS unit in a vibrating or stimulating assembly may generate a waveform having a pulse frequency of 2 to 150 pulse per seconds (PPS). In embodiments, a TENS unite may generate a waveform having a pulse width duration of 50 to 250 microseconds. In embodiments, a vibrating or stimulating assembly 570 may comprise a housing 572. In embodiments, the housing 572 may comprise a microprocessor or controller 571, a signal generator 575 and an array of one or more electrodes 573. In some embodiments, the signal generator 575 may be coupled or connected to the array of one or more electrodes through one or more wires. In some embodiments, the electrodes may appear to be patches. In some embodiments, the signal transmitted from the signal generating device 510 is communicated through a wire or cable 525 to the microprocessor or controller 571. In some embodiments, the microprocessor or controller 571 generates instructions and/or messages which are communicated to a signal generator 575. In some embodiments, the signal generator 575 may generate biphasic, symmetrical, or rectangular pulses with a regulated current. In some embodiments, other technical specifications (such as a pulse waveform shape, a maximum output voltage, a maximum output current, and pulse pattern) have been contemplated and are considered to be within the scope of the claimed subject matter. In some embodiments, the attributes of the signal generator 575 may be programmable, with the microprocessor 572 and/or signal generator 575 being connectable (such as through the USB port) to a computer or mobile device (e.g., smart phone, tablet computer, etc.) running appropriate setup software. In this fashion, the attributes of signal generator 575 may be customized to the user's pain characteristics, physiology, and preferences. In some embodiments, the signal generator 575 may communicate the generated signals with the selected attributes to the array of one or more electrodes 573. In embodiments, the electrodes 573 may be pressed against the housing 571 which is pressed against the gum or tissue of the patient. In embodiments, the housing 571 may have a fabric covering, a plastic covering and/or a composite covering. In some embodiments, the one or more electrodes 573 may not be included in the housing 570 and may stick out or protrude from the housing 570.

FIG. 5D illustrates a vibrating or stimulation assembly utilizing an ultrasound unit or device to stimulate a gum area or a tissue area according to embodiments. In embodiments, a stimulation assembly 580 may comprise a housing 581. In some embodiments, a housing 581 may include a controller or processor 582, an ultrasound generator 583 and/or one or more transducers 584. In some embodiments, a signal transmitted from the signal generating device 510 may be communicated through a wire or cable 525 to a controller or processor 582 in a stimulation assembly 580. In embodiments, computer-readable instructions executable by a controller or processor 582 may communication instructions, commands or messages including respective parameters to an ultrasound waveform generator 583 which in turn will generate an ultrasound waveform. In some embodiments, an ultrasound waveform may have a frequency of 1 Megahertz with a 20 percent duty cycle pulse. In some embodiments, an ultrasound waveform may have a frequency of between 25 kilohertz to 1 Megahertz. In some embodiments, an ultrasound waveform may have an intensity of 1 Watt per square centimeter. In some embodiments, an ultrasound waveform may have a 100-Hz frequency. In some embodiments, the ultrasound generator 583 may communicate the generated ultrasound to the one or more transducers 584. In some embodiments, the one or more transducers 584 may be positioned against a patient's tissue or gum to stimulate the tissue or gum. In some embodiments, the one or more transducers 584 may be moved in a circular motion in order to more effectively utilize the ultrasound transducer 584. In some embodiments, a gel or lotion may be applied to the area of the gum to which the ultrasound transducer may be applied. In some embodiments, a housing 581 may have an opening where the one or more transducers 584 may stick out from and/or exit from. In embodiments, there may be no covering, skin or fabric on the one or more transducers 584 which may be applied directly to a skin, tissue or gum. In embodiments, a gel or disinfectant may be utilized to sterilize the one or more transducers 584.

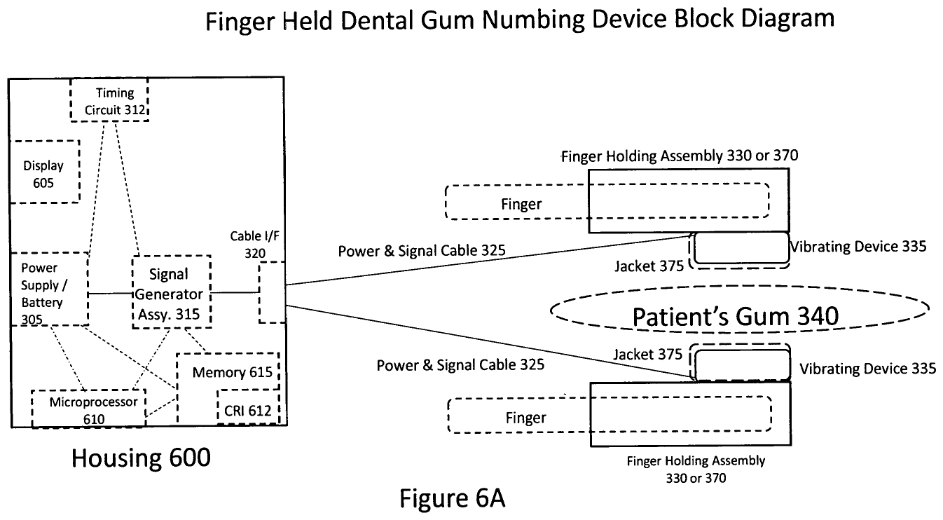

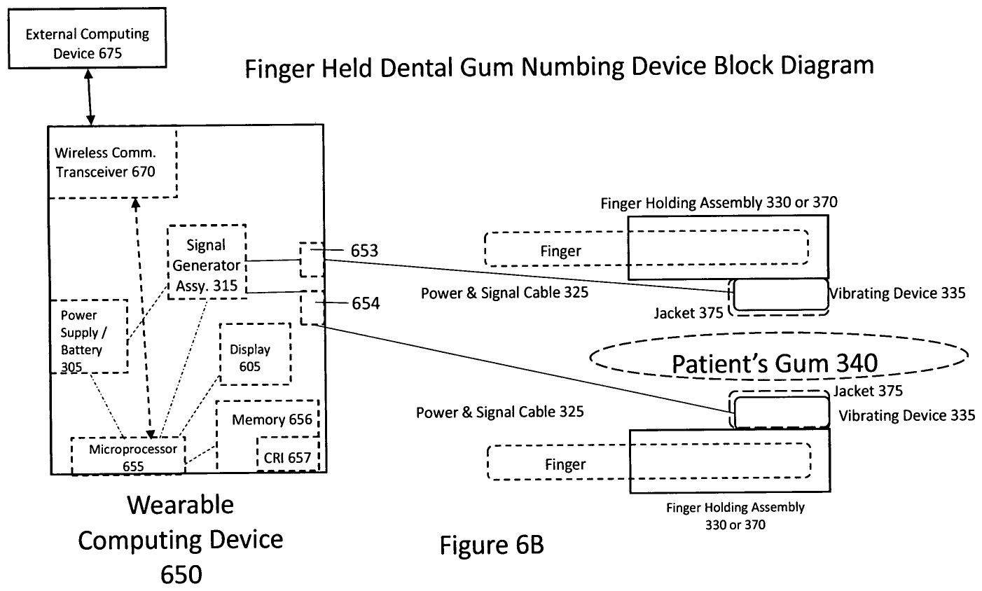

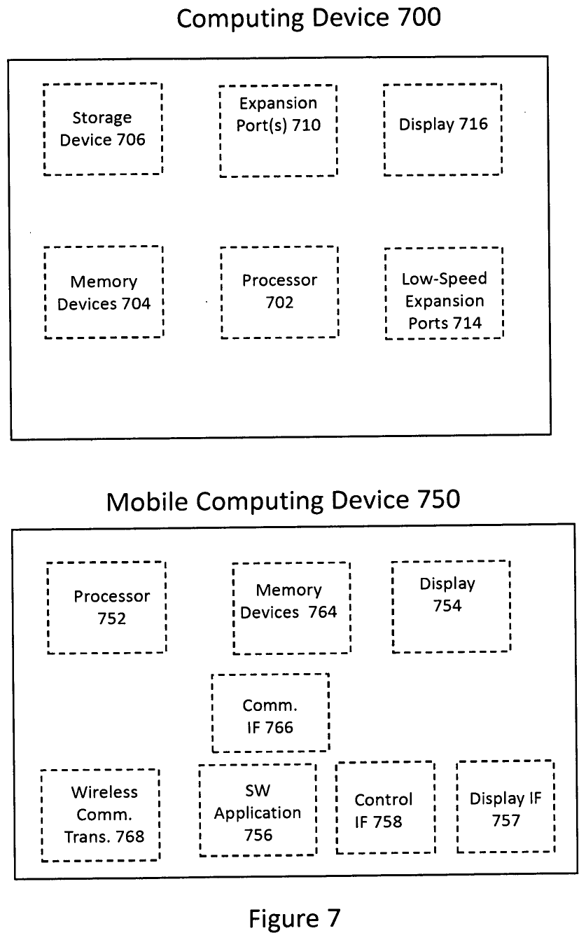

FIG. 6A illustrates a block diagram of an embodiment of an electronics housing including a display according to some embodiments. In some embodiments, FIG. 6A recites a different configuration of an electronics housing according to some embodiments. In some embodiments, the electronics housing 600 may comprise one or more microprocessors 610, one or more memory devices 615, computer readable instructions 612, one or more displays 605, a power supply or battery 305, a signal generator or assembly 315, an output port 320 (e.g., or cable interface), and/or a timing circuit 312. In some embodiments, the signal generator assembly 315, the power supply/battery 305, and/or the timing circuit 312 may operate as described above with respect to FIG. 3. In the embodiments illustrated in FIG. 6A, the housing 600 may include computer-readable instructions 612 stored in the one or more memory devices 615 and and/or executable by one or more processors 610 to perform many functions of the gum numbing system. In some embodiments, the housing 600 may comprise a display 605. In some embodiments, the display 605 may be a touchscreen display. In some embodiments, the display 605 may show status of certain operations or events (e.g., whether the electronics housing is communicating a signal to the two or more gum stimulation or vibrating devices 335). In some embodiments, a medical professional may utilize the touchscreen display 605 to perform certain actions. In some embodiments, when a medical professional swiped on certain parts of the display 605, computer-readable instructions 612 may be executable by the one or more microprocessors 610 to, for example, turn on or off the power supply or battery 305, turn on or off the signal generator assembly 315, adjust an amplitude, frequency and/or period of a signal that is transmitted by the signal generator assembly 315 to the two or more vibrating or stimulations assemblies 335, and/or set a time for the timing circuit 312 to operate. This minimizes the amount of other electrical and/or mechanical components that are utilized by or included in the electronics housing 600. In some embodiments, a display manager may coordinate information on a touch screen display. In some embodiments, the display manager may coordinate data received from various different applications or module or input devices. In some embodiments, the electronics housing 600 may include an accelerometer, which may determine a state of the device or housing (e.g., whether it has been moved or to recognize gestures and/or body movements). In some embodiments, the accelerometer may cooperate with other input devices. In some embodiments, device inputs from a touch screen may be processed by an input manager and the input manager may forward the information to different applications or modules. In some embodiment, the input manager may process gesture-based inputs received from input devices such as sensor-emitter pairs.

In some embodiments, the power source of all of the devices described herein may comprise an AC/DC power supply that can convert an AC signal to a DC voltage signal that meets the operating requirement of the device components. In some embodiments, the DC power supply that can convert to a DC voltage signal that meets the operating requirement of the device components. In some embodiments, the power source may be charged through contact or non-contact charging.

FIG. 6B illustrates a wearable computing device interfacing with two or more gum stimulation or vibrating devices according to some embodiments. The advent of wearable computing devices (e.g., watches, exercise devices (Fitbit), and/or potentially computing glasses) may also be utilized with two or more gum stimulation assemblies 335 in order to minimize pain for patients when needles are inserted into a patient's gum. In these embodiments, the smart watch or exercise bands (e.g., Fitbit) may communicate signals and/or transmit voltages and/or currents to the two or more gum stimulation assemblies 335 in order to communicate with and/or activate the two or more gum stimulation assemblies. In some embodiments, the wearable computing devices 650 may include additional output ports or interfaces to which wires and/or cables could be connected in order to transmit the signal and/or voltage and/or current to the two or more gum stimulation accessories. This provides the additional advantage of utilizing an existing hardware device (e.g., the wearable computing device 650) and adding on unique functionality that may be specifically utilized in the dentist industry without having to create a separate electronics housing that has to be worn on a medical professional's wrist. In addition, an external computing device (e.g., a mobile communications device, smartphone, tablet, laptop or desktop computing device) 675 may communicate with wearable computing device 650 and/or may also provide additional functionality for the gum vibration or stimulation devices.

FIG. 6B illustrates the wearable computing device 650 interfacing and communicating with the two or more gum vibrating and stimulating devices or assemblies 335. In some embodiments, the wearable computing device 650 may include all of its existing functionality (e.g., smart watch, exercise device (e.g., Fitbit), or other device). In addition, the wearable computing device 650 may further comprise one or more output ports or interfaces to provide a signal or a DC voltage and/or current to the vibrating devices 335. In the embodiment illustrated in FIG. 6B, the wearable computing device 650 may comprise two output ports 653 and 654 to communicate the signal and/or DC voltage and/or current to the two or more vibrating devices 335 (via, for example, the power and signal cables). The two or more vibrating or stimulating devices 335 may operate in similar fashions to the devices described in FIGS. 3, 4A-4E, and/or 5A-5D. In some embodiments, the wearable computing device may comprise one or more wireless communication transceivers 670, one or more power supply/battery 305, one or more signal generator or signal generation assembly 315, one or more display 605, one or more processors or controllers 655, one or more memory devices 656 and/or computer-readable instructions 657. In some embodiments, the computer-readable instructions 657 may be stored in one or more memory devices 656 and may be executable by one or more processors to perform certain actions and/or operations of the wearable computing device 650 as well as the actions necessary to perform operations of the gum numbing system (e.g., activating the signal generator assembly or circuity 315 and/or communicating a signal, DC voltage and/or DC current to the two or more vibrating devices 335). In some embodiments, the display 605 may be a touchscreen display. In some embodiments, a graphical user interface on the display 605 may receive user (e.g., medical professional) input in order to perform the operations. In some embodiments, the graphical user interface on the display 605 may receive an input to turn on or activate the two or more vibrating devices 335 on the touchscreen. In some embodiments, the computer-readable instructions 657 may be executable by the one or more processors 655 to activate or turn on the signal generator 315. In some embodiments, the signal generator 315 may communicate a signal and/or DC voltage and/or current to the output ports 653 and 654 to be communicated or transmitted to the two or more vibrating devices 335. In some embodiments, an external computing device 675 (e.g., a software application on the external computing device 675) may communicate with the wearable computing device 675 via the one or more wireless communication transceivers 670 (e.g., Bluetooth transceiver, WiFi transceiver, cellular transceiver). In some embodiments, this allows medical professionals utilizing other computing devices 675 to communicate with the wearable computing device 650 and receive status and/or operating parameters from the two or more vibrating devices 335.

In some embodiments, the wearable computing device 650 may also include knobs or buttons on a side of the wearable computing device 650. In some embodiments, the knobs or buttons may be used as input devices to allow a use to scroll through screens, adjust a magnification of a display 605, go to a home or initial screen and/or to activate accessories including the signal generator 315 to then transmit or communicate the signal, DC voltage and/or DC current. In some embodiments, the knobs and/or buttons may be associated with or programmed to have certain operations or functions activated or performed. In some embodiments, two buttons or clasps may be utilized to attach the wristband and/or watchband of the wearable computing device 650. In some embodiments, the touchscreen may be a flexible retina display or another hard material in order to resist scratches and/or impact. In some embodiments, the display may include touch sensors or electrodes in order to sense not only that a certain area is being selected but also to detect a force being utilized. In some embodiments, the wearable computing device 650 may also include one or more accelerometers and/or gyroscopes to detect movement of the wearable computing device 650 and/or other parts of the body of the user or operator wearing the wearable computing device 650. In some embodiments, the wearable computing device 650 may include a linear actuator and/or a speaker that provides haptic feedback or gives tactile cues when specific actions are occurring (e.g., such as the signal generator is activated to deliver signals or DC voltages or currents to the gum stimulating devices. In some embodiments, wearable computing device may include one or more wireless communication transceivers. In some embodiments, these wireless communication transceivers may include a WiFi 82.11b/g, other 802.11 transceivers, personal area network transceivers, cellular transceivers and/or a near field communication transceivers. In some embodiments, the wearable computing device 650 may also include a camera, a speaker and/or microphone.

FIG. 6 shows an example of a computer device 700 and a mobile computer device 750, which may be used with the techniques or devices described herein. Computing device 700 is intended to represent various forms of digital computers, such as laptops, desktops, workstations, personal digital assistants, servers, cloud-based servers, networks, mainframes, and other appropriate computers. Computing device 750 is intended to represent various forms of mobile devices, such as personal digital assistants, cellular telephones, smartphones, mobile communication devices, wearable computing devices and other similar computing devices. The components shown here, their connections and relationships, and their functions, are meant to be exemplary only, and are not meant to limit implementations of the inventions described and/or claimed in this document.

Computing device 700 includes one or more processors 702, one or more memory devices 704, one or more other storage devices 706, a high-speed bus or other interface connecting to the one or more memory devices 704 and/or one or more high-speed expansion ports 710, and a low speed interface connecting to a low speed bus or interface and/or one or more other storage devices 706. Each of the components 702, 704, 706, and 710, may be interconnected using various busses, and may be mounted on a common motherboard or in other manners as appropriate. The one or more processors 702 can process instructions for execution within the computing device 700, including computer-readable instructions stored in the one or more memory devices 704 or on the one or more other storage devices 706 to display graphical information for a GUI on an external input/output device, such as display 716 coupled to a high speed interface. In other implementations, multiple processors and/or multiple buses may be used, as appropriate, along with multiple memories and types of memory devices. Also, multiple computing devices 700 may be connected, with each device providing portions of the necessary operations (e.g., as a server bank, a group of cloud-based servers, or a multi-processor system).

In some embodiments, the one or more memory devices 704 may store information or computer-readable instructions within the computing device 700. In some embodiments, the one or more memory devices 704 may be a volatile memory device. In some embodiments, the one or more memory devices 704 may be a non-volatile memory device. The one or more memory devices 704 may also be another form of computer-readable medium, such as a magnetic or optical disk, a random access memory, a dynamic random access memory, and/or one or more flash memory devices.

In some embodiments, the one or more other storage devices 706 may be capable of providing mass storage for the computing device 700. In some embodiments, the one or more other storage devices 706 may be or contain a computer-readable medium, such as a floppy disk device, a hard disk device, an optical disk device, or a tape device, a flash memory or other similar solid state memory device, or an array of devices, including devices in a storage area network or other configurations. In some embodiments, a computer program product can be tangibly embodied in an information carrier. In some embodiments, the computer program product may also contain instructions that, when executed, perform one or more methods, such as those described above.

In some embodiments, a high-speed controller may manage bandwidth-intensive operations for the computing device 700, while the low speed controller may manage lower bandwidth-intensive operations. In some embodiments, the high-speed controller may be coupled to the one or more memory devices 704, display 716 (e.g., through a graphics processor or accelerator), and to high-speed expansion ports 710, which may accept various expansion cards (not shown). In some embodiments, low-speed controller may be coupled to one or more other storage devices 706 and low-speed expansion port 714. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

In some embodiments, computing device 750 may include one or more processors 752, one or more memory devices 764, an input/output device such as a display 754, one or more communication interfaces 766, and one or more wireless communication transceivers 768, among other components. In some embodiments, the computing device 650 may also be provided with a storage device, such as a microdrive or other device, to provide additional storage. In some embodiments, the components 750, 752, 764, 754, 766, and 768, may be interconnected using various buses or other communication interfaces, and several of the components may be mounted on a common motherboard or in other manners as appropriate.

In some embodiments, the one or more processors 752 can execute instructions within the computing device 750, including computer-readable instructions stored in the one or more memory devices 764. In some embodiments, the one or more processors may be implemented as a chipset of chips that include separate and multiple analog and digital processors. In some embodiments, the one or more processors 752 may provide, for example, for coordination of the other components of the computing device 750, such as control of user interfaces, software applications 756 run by device 750, and wireless communication by computing device 750.

In some embodiments, the one or more processors 752 may communicate with a user through control interface 758 and display interface 757 coupled to a display 754. In some embodiments, the display 754 may be, for example, a TFT LCD (Thin-Film-Transistor Liquid Crystal Display) or an OLED (Organic Light Emitting Diode) display, or other appropriate display technology. In some embodiments, the display interface 757 may comprise appropriate circuitry for driving the display 754 to present graphical and other information to a user. In some embodiments, the control interface 758 may receive commands from a user and convert them for submission to the one or more processors 752. In addition, an external interface may be provided in communication with the one or more processors 752, so as to enable near area communication of device 750 with other devices. External communication interface 766 may provide, for example, for wired communication in some implementations, or for wireless communication in other implementations, and multiple interfaces may also be used.

In some embodiments, the one or more memory devices 764 may store information or computer-readable instructions within the computing device 750. In some embodiments, the one or more memory devices 764 can be implemented as one or more of a computer-readable medium or media, a volatile memory device or devices, or a non-volatile memory device or devices. In some embodiments, expansion memory may also be provided and connected to computing device 750 through an expansion interface, which may include, for example, a SIMM (Single In Line Memory Module) card interface. In some embodiments, such expansion memory may provide extra storage space for computing device 750, or may also store applications or other information for computing device 750. Specifically, expansion memory may include instructions to carry out or supplement the processes described above, and may include secure information also. Thus, for example, expansion memory 674 may be provide as a security module for device 750, and may be programmed with instructions that permit secure use of computing device 750. In addition, secure applications may be provided via the SIMM cards, along with additional information, such as placing identifying information on the SIMM card in a non-hackable manner.

The one or more memory devices may be for example, flash memory and/or NVRAM memory. In some embodiments, a computer program product is tangibly embodied in an information carrier. The computer program product contains computer-readable instructions that, when executed, perform one or more methods, such as those described above. In some embodiments, the information carrier may be a computer- or machine-readable medium, such as the one or more memory devices 764, expansion memory, memory on the one or more processors 752, or a propagated signal that may be received, for example, over wireless communication transceiver 768 or communication interface 766.

Computing device 750 may communicate wirelessly through wireless communication transceivers 768, which may include digital signal processing circuitry where necessary. In some embodiments, wireless communication interface 768 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through one or more radio-frequency transceivers. In addition, in some embodiments, short-range communication may occur, such as using a Bluetooth, WiFi, or other such transceiver (not shown). In addition, in some embodiments, GPS (Global Positioning System) receiver module may provide additional navigation- and location-related wireless data to computing device 750, which may be used as appropriate by applications running on computing device 750. In some embodiments, computing device 750 may also communicate audibly using audio receivers or chipsets or microphones, which may receive spoken information from a user and convert it to usable digital information. Audio receivers or chipsets or microphones may likewise generate audible sound for a user, such as through a speaker, e.g., in a handset of computing device 650. Such sound may include sound from voice telephone calls, may include recorded sound (e.g., voice messages, music files, etc.) and may also include sound generated by applications operating on computing device 750.

Different embodiments and/or implementations of the systems and techniques described here can be realized in digital electronic circuitry, integrated circuitry, specially designed ASICs (application specific integrated circuits), computer hardware, firmware, software, and/or combinations thereof. These different embodiments and/or implementations may include implementations or embodiments in one or more computer programs (e.g., computer-readable instructions) that are executable and/or interpretable on a programmable device, including one or more programmable processors, which may be special or general purpose, coupled to receive data and instructions from, and to transmit data and instructions to, one or more storage systems, one or more input devices, and one or more output devices.

In some embodiments, these computer programs (also known as programs, software, software applications or code) may include computer-readable instructions for one or more programmable processors, and may be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to the one or more programmable processors, including a machine-readable medium that receives machine instructions as a machine-readable signal.

To provide for interaction with a user, the systems and techniques described here can be implemented on a computing device having a display device (e.g., a CRT (cathode ray tube) or LCD (liquid crystal display) monitor) (or other display devices) for displaying information to the user and a keyboard, a touchscreen, and a pointing device (e.g., a mouse or a trackball), or one or more microphones and/or audio processing circuitry (e.g., codecs) by which the user can provide input to the computing device. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

In some embodiments, the systems and techniques described here can be implemented in a computing system that includes a back end component (e.g., as a data server or a database server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

In some embodiments, the computing system can include client devices and/or server devices. In some embodiments, a client device and/or server device are generally remote from each other (and/or not in the same location) and typically may interact through a communication network (e.g., a wired communication network or a wireless communication network). In some embodiments, the relationship of the client device and server device arises by virtue of computer programs running on the respective computing device and having a client-server relationship to each other.

In some embodiments, a gum numbing system may include a wristband configured to be attached to a user' wrist; an electronics housing; two or more finger coverings; two or more gum stimulation assemblies, the two or more gum stimulation assemblies detachably connected to the two or more finger coverings and the two or more gum stimulation assemblies to stimulate a patient's gum before a needle is inserted into the patient's gum; and two or more cables or wires, the two or more cables or wires connecting the electronics housing to the two or more gum stimulation assemblies. In some embodiments, the electronics housing may include a rechargeable power source, one or more signal generator or control circuits; and/or one or more cable or wire interface ports, wherein the two or more cables or wires are connected to the electronics housing via the one or more cable or wire interface ports and wherein the signal generator or control circuitry generates a signal to activate or deactivate the two or more gum stimulation assemblies.

In some embodiments, the gum numbing system further includes a switching assembly, the switching assembly coupled to the rechargeable power source, the switching assembly to activate or deactivate the signal generator or control circuitry. In some embodiments, the gum numbing system includes an adjustment assembly, the adjustment assembly coupled to the signal generator or control circuitry, the adjustment assembly to adjust a frequency and/or an amplitude of a signal transmitted by the signal generator, the adjustment assembly to comprise a knob or an adjustment dial. In some embodiments, the gum numbing system further includes an activation assembly, the activation assembly coupled to the rechargeable power source or the signal generator or control circuitry, the activation assembly configurable to turn on or off the rechargeable power source or the signal generator to activate or deactivate the gum numbing system.

In some embodiments, the two or more gum stimulation assemblies may include a motor, a shaft and a cap, the motor coupled to the shaft and the shaft coupled to the cap, wherein the motor is configurable to receive a signal from electronics housing through the two or more cables to activate the motor, the activation of the motor to rotate the shaft and the cap, and the rotation of the shaft and the cap to cause the two or more gum stimulation assemblies to vibrate. In some embodiments, the two or more gum stimulation assemblies further include a motor controller, wherein the motor controller is configurable to receive a signal from the electronics housing through the two or more cables to control the motor and the motor controller to communicate the signal to the motor. In some embodiments, a gum stimulation assembly comprises one or more inducting coils, one or more resistors and one or more capacitors, the gum stimulation assembly to receive a signal from the electronics housing via a cable of the two or more cables, the signal to be filtered by the one or more resistors and the one or more capacitors, the filtered signal to be received by the one or more inducting coils, the one or more inducting coils to generate an oscillating magnetic field, the oscillating magnetic field to stimulate the patient's gums. In some embodiments, the gum stimulation assembly includes one or more processors, one or more signal generators, and one or more electrodes, the one or more processors to receive a signal from the electronics housing, the one or more processors to communicate commands or instructions to the one or more signal generators, the one or more signal generators to generate biphasic, symmetrical or rectangular pulses with a regulated current to the one or more electrodes, the one or more electrodes to apply a signal to the patient's gums. In some embodiments, the gum stimulation assembly includes one or more processors, an ultrasound signal generator, and one or more transducers, the one or more processors to receive a signal from the electronics housing, the one or more processors to communicate commands or instructions to the ultrasound signal generators, the ultrasound signal generator to generate an ultrasound signal to the one or more transducers, the one or more transducers to press against the patient's bum to stimulate the patient's gums.

A number of embodiments have been described. Nevertheless, it will be understood that various modifications may be made without departing from the spirit and scope of the invention. For example, much of this document has been described with respect to a wristwatch form factor, but other forms of devices may be addressed.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

The above disclosure is sufficient to enable one of ordinary skill in the art to practice the invention, and provides the best mode of practicing the invention presently contemplated by the inventor. While there is provided herein a full and complete disclosure of the preferred configurations of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like. The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A gum numbing system, comprising:
a wristband configured to be attached to a user' wrist;
an electronics housing;
two or more finger coverings;
two or more gum stimulation assemblies, the two or more gum stimulation assemblies detachably connected to the two or more finger coverings and the two or more gum stimulation assemblies to stimulate a patient's gum before a needle is inserted into the patient's gum; and
two or more cables or wires, the two or more cables or wires connecting the electronics housing to the two or more gum stimulation assemblies.

2. The gum numbing system of claim 1, wherein the electronics housing is integrated into the wristband.

3. The gum numbing system of claim 1, wherein the electronics housing is attached to a top surface of wristband.

4. The gum numbing system of claim 1, the electronics housing including a rechargeable power source, one or more signal generator or control circuits; and/or one or more cable or wire interface ports, wherein the two or more cables or wires are connected to the electronics housing via the one or more cable or wire interface ports and wherein the signal generator or control circuitry generates a signal to activate or deactivate the two or more gum stimulation assemblies.

5. The gum numbing system of claim 4, the electronics housing further include a power input port (e.g., a USB connector), the power input port to receive power from an external power source and to charge the rechargeable power source.

6. The gum numbing system of claim 5, the electronics housing configured to be detachable from the wristband to allow the electronics housing, and the rechargeable power source, to be chargeable separately from the remainder of the gum numbing system.

7. The gum numbing system of claim 6, the electronics housing including one or more inductive charging coils, the one or more inductive charging coils to receive power from a induction charging plate.

8. The gum numbing system of claim 4, further comprising a switching assembly, the switching assembly coupled to the rechargeable power source, the switching assembly to activate or deactivate the signal generator or control circuitry.

9. The gum numbing system of claim 4, further comprising an adjustment assembly, the adjustment assembly coupled to the signal generator or control circuitry, the adjustment assembly to adjust a frequency and/or an amplitude of a signal transmitted by the signal generator, the adjustment assembly to comprise a knob or an adjustment dial.

10. The gum numbing system of claim 4, further comprising an activation assembly, the activation assembly coupled to the rechargeable power source or the signal generator or control circuitry, the activation assembly configurable to turn on or off the rechargeable power source or the signal generator to activate or deactivate the gum numbing system.

11. The gum numbing system of claim 1, further comprising two or more shells, the two or more shells covering the associated two or more gum stimulation assemblies.

12. The gum numbing system of claim 1, wherein the two or more gum stimulation assemblies further comprise a motor, a shaft and a cap, the motor coupled to the shaft and the shaft coupled to the cap, wherein the motor is configurable to receive a signal from electronics housing through the two or more cables to activate the motor, the activation of the motor to rotate the shaft and the cap, and the rotation of the shaft and the cap to cause the two or more gum stimulation assemblies to vibrate.

13. The gum numbing system of claim 12, further comprising two or more finger coverings, the finger coverings to cover a medical professional's fingers, the finger coverings to couple to the two or more gum stimulation assemblies and to allow the user to control placement of the two or more gum stimulation assemblies in a patient's mouth.

14. The gum numbing system of claim 13, the two or more finger coverings to comprise an adhesive layer, the adhesive layer to attach the two or more finger coverings to the two more gum stimulation assemblies.

15. The gum numbing system of claim 1, wherein two or more gum stimulation assemblies further comprise a motor controller, a motor, a shaft and a cap, the motor controller coupled to the motor, the motor coupled to the shaft and the shaft coupled to the cap, wherein the motor controller is configurable to receive a signal from the electronics housing through the two or more cables to control the motor, the motor controller to communicate the signal to the motor, wherein the motor rotates the shaft and the cap, and the rotation of the shaft and the cap to cause the two or more gum stimulation assemblies to vibrate.

16. The gum numbing system of claim 1, wherein a gum stimulation assembly comprises one or more inducting coils, one or more resistors and one or more capacitors, the gum stimulation assembly to receive a signal from the electronics housing via a cable of the two or more cables, the signal to be filtered by the one or more resistors and the one or more capacitors, the filtered signal to be received by the one or more inducting coils, the one or more inducting coils to generate an oscillating magnetic field, the oscillating magnetic field to stimulate the patient's gums.

17. The gum numbing system of claim 1, wherein a gum stimulation assembly comprises one or more processors, one or more signal generators, and one or more electrodes, the one or more processors to receive a signal from the electronics housing, the one or more processors to communicate commands or instructions to the one or more signal generators, the one or more signal generators to generate biphasic, symmetrical or rectangular pulses with a regulated current to the one or more electrodes, the one or more electrodes to apply a signal to the patient's gums.

18. The gum numbing system of claim 1, wherein a gum stimulation assembly comprises one or more processors, an ultrasound signal generator, and one or more transducers, the one or more processors to receive a signal from the electronics housing, the one or more processors to communicate commands or instructions to the ultrasound signal generators, the ultrasound signal generator to generate an ultrasound signal to the one or more transducers, the one or more transducers to press against the patient's bum to stimulate the patient's gums.
